SEQUENCE LISTING (4 sequences)

SEQ ID NO: 1 - protein, 440 aa, Artificial Sequence
  RT p51
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKQKKSVTVLDVGDAYFSVPLDKDFRKYTAFTIPSINNETPGIRYQYNVLPQGWKGSPAIFQCSMTKILEPFRKQNPDIVIYQYMDDLYVGSDLEIGQHRTKIEELRQHLLRWGFTTPDKKHQKEPPFLWMGYELHPDKWTVQPIVLPEKDSWTVNDIQKLVGKLNWASQIYAGIKVRQLCKLLRGTKALTEVVPLTEEAELELAENREILKEPVHGVYYDPSKDLIAEIQKQGQGQWTYQIYQEPFKNLKTGKYARMKGAHTNDVKQLTEAVQKIATESIVIWGKTPKFKLPIQKETWEAWWTEYWQATWIPEWEFVNTPPLVKLWYQLEKEPIIGAETF

SEQ ID NO: 2 - protein, 560 aa, Artificial Sequence
  RT p66
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKQKKSVTVLDVGDAYFSVPLDKDFRKYTAFTIPSINNETPGIRYQYNVLPQGWKGSPAIFQCSMTKILEPFRKQNPDIVIYQYMDDLYVGSDLEIGQHRTKIEELRQHLLRWGFTTPDKKHQKEPPFLWMGYELHPDKWTVQPIVLPEKDSWTVNDIQKLVGKLNWASQIYAGIKVRQLCKLLRGTKALTEVVPLTEEAELELAENREILKEPVHGVYYDPSKDLIAEIQKQGQGQWTYQIYQEPFKNLKTGKYARMKGAHTNDVKQLTEAVQKIATESIVIWGKTPKFKLPIQKETWEAWWTEYWQATWIPEWEFVNTPPLVKLWYQLEKEPIIGAETFYVDGAANRETKLGKAGYVTDRGRQKVVPLTDTTNQKTELQAIHLALQDSGLEVNIVTDSQYALGIIQAQPDKSESELVSQIIEQLIKKEKVYLAWVPAHKGIGGNEQVDGLVSAGIRKVL

SEQ ID NO: 3 - protein, 440 aa, Artificial Sequence
  RT p51_2
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETF

SEQ ID NO: 4 - protein, 560 aa, Artificial Sequence
  RT p66_2
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVL

(19) United States
(12) Patent Application Publication
(10) Pub. No.: US 2024/0018492 A1
(43) Pub. Date: Jan. 18, 2024

(54) PRIME EDITING USING HIV REVERSE TRANSCRIPTASE AND CAS9 OR VARIANT THEREOF

(71) Applicant: INSTITUTE FOR BASIC SCIENCE, Daejeon (KR)

(72) Inventors: Jin-Soo KIM, Seoul (KR); Chul-Yong PARK, Daejeon (KR)

(21) Appl. No.: 18/247,108

(22) PCT Filed: Sep. 29, 2021
(86) PCT No.: PCT/KR2021/013326

§ 371 Date: Mar. 29, 2023

(30) Foreign Application Priority Data
Sep. 29, 2020 (KR) 10-2020-0126709

(51) Int. Cl. C12N 9/22; C12N 15/10; C12N 15/63; C12N 9/12

(52) U.S. Cl. CPC C12N 9/22; C12N 15/102; C12N 15/63; C12N 9/1276; C12N 2310/20; C12Y 207/07049

(57) ABSTRACT

The present invention relates to: a prime editing composition comprising a prime editor protein or a nucleic acid encoding same, and a prime editing guide RNA (pegRNA); and a prime editing method.

### TECHNICAL FIELD

The present invention relates to a composition for prime editing including a prime editor protein or a nucleic acid encoding the same and a prime editing guide RNA (pegRNA), and a prime editing method.

### BACKGROUND ART

In order to overcome the limitations of flexibility and precision in gene editing by CRISPR, which includes a molecular complex configured such that a guide DNA that recognizes a specific site in the genome is coupled with a Cas9 enzyme that cleaves the double helix of DNA, improved genome editing methods have been reported.

Specifically, a prime editing method has been reported using a prime editor protein complex composed of nickase Cas9 (H840A) and M-MLV reverse transcriptase, in which nickase Cas9 induces modification to cleave only one DNA strand, reverse transcriptase copies one RNA template to create new DNA, and a prime editing guide RNA (pegRNA) directs the prime editor protein complex to the target site to edit the genome (Anzalone A V, Randolph P B, Davis J R et al., “Search-and-replace genome editing without double-strand breaks or donor DNA,” Nature. 2019 Oct. 21).

A conventional prime editor is provided in a form in which Moloney murine leukemia virus (M-MLV) reverse transcriptase is linked to nickase Cas9 (H840A), but the MMLV-based reverse transcriptase is disadvantageous in that it is difficult to insert long genetic information.

Against this technical background, the inventors of the present application ascertained that efficient prime editing is possible using HIV RT instead of MMLV, thus culminating in the present invention.

### DISCLOSURE

It is an object of the present invention to provide a composition for prime editing including a prime editor protein or a nucleic acid encoding the same and a prime editing guide RNA (pegRNA).

It is another object of the present invention to provide a prime editing method using a prime editor protein or a nucleic acid encoding the same and a prime editing guide RNA (pegRNA).

In order to accomplish the above objects, the present invention provides a composition for prime editing including a prime editor protein or a nucleic acid encoding the same and a prime editing guide RNA (pegRNA), in which the prime editor protein includes (i) a target-specific nuclease or a variant thereof and (ii) an HIV (human immunodeficiency virus) reverse transcriptase (RT) or a variant thereof.

In addition, the present invention provides a prime editing method including treating cells with the composition described above.

### DESCRIPTION OF DRAWINGS

FIG. 1 shows results confirming the editing efficiency of an HIV reverse transcriptase-based prime editor (PE) as phenotype in a human cell line (HEK-293 GFP stable cell) using a composition according to the present invention; and

FIG. 2 shows results confirming the CTT-to-GCC editing efficiency induced by the HIV reverse transcriptase-based prime editor in the human cell line (HEK-293 GFP stable cell) using the composition according to the present invention at the genome level by NGS (next generation sequencing).

### MODE FOR INVENTION

Unless otherwise defined, all technical and scientific terms used herein have the same meanings as typically understood by those skilled in the art to which the present invention belongs. In general, the nomenclature used herein is well known in the art and is typical.

The inventors of the present application found that, in a conventional prime editor in which MMLV (Moloney murine leukemia virus) reverse transcriptase is linked to SpCas9 nickase (H840A), MMLV reverse transcriptase acts as a monomer, but it is difficult to insert long genetic information.

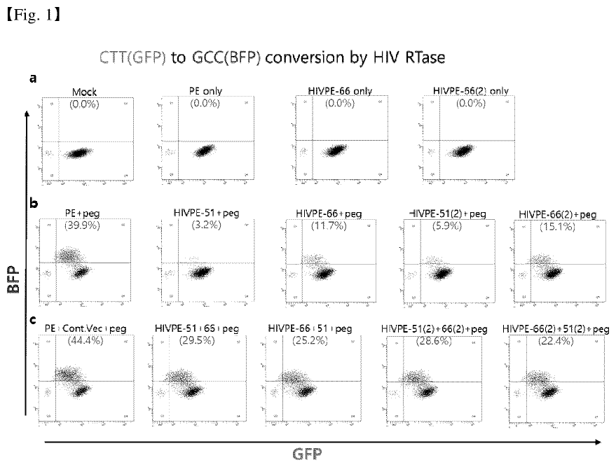

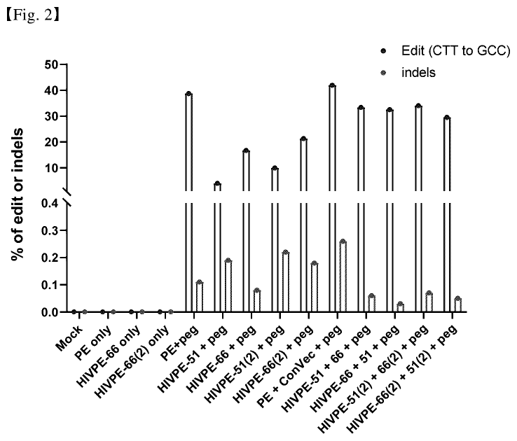

Based thereon, it was confirmed that there is an efficient prime editing effect in a human cell line through the prime editor protein constructed including an MMLV reverse transcriptase or a variant thereof (FIGS. 1 and 2).

Based thereon, an aspect of the present invention pertains to a composition for prime editing including a prime editor protein or a nucleic acid encoding the same and a prime editing guide RNA (pegRNA), in which the prime editor protein includes (i) a target-specific nuclease or a variant thereof and (ii) an HIV (human immunodeficiency virus) reverse transcriptase (RT) or a variant thereof.

Another aspect of the present invention pertains to a prime editing method including treating cells with the composition.

As used herein, the term “editing” may be used interchangeably with “proofreading” and refers to a method of altering a nucleic acid sequence by selective deletion of a specific genomic target. Such specific genomic targets include, but are not limited to, chromosomal regions, genes, promoters, open reading frames, or any nucleic acid sequence.

As used herein, the term “target” or “target site” refers to a previously identified nucleic acid sequence of any composition and/or length. Such target sites include, but are not limited to, chromosomal regions, genes, promoters, open reading frames, or any nucleic acid sequence.

As used herein, the term “on-target” refers to a subsequence of a specific genomic target that may be perfectly complementary to a programmable DNA-binding region and/or a single guide RNA sequence.

As used herein, the term “off-target” refers to a subsequence of a specific genomic target that may be partially complementary to a programmable DNA-binding region and/or a single guide RNA sequence.

The nuclease may be target-specific, and examples thereof may include, but are not limited to, ZFN (zinc finger nuclease), TALEN (transcriptional activator-like effector nuclease), and Cas protein. The target-specific nuclease or the variant thereof may be a Cas protein or a variant thereof. The nuclease variant may be mutated to eliminate endonuclease activity of cleaving double-stranded DNA in the nuclease and have nickase activity. The nuclease variant may be, for example, a variant of Cas9.

The Cas protein may include, but is not limited to, an endonuclease of Cast, Cas1B, Cast, Cas3, Cas4, Cas5, Cas6, Cas7, Cas8, Cas9, Cas10, Cas12a, Cas12b, Cas12c, Cas12d, Cas12e, Cas12g, Cas12h, Cas12i, Cas12j, Cas13a, Cas13b, Cas13c, Cas13d, Cas14, Csy1, Csy2, Csy3, Cse1, Cse2, Csc1, Csc2, Csa5, Csn2, CsMT2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, or Csf4, especially Cas9.

The Cas protein is a major protein component of the CRISPR/Cas system and is a protein capable of forming an activated endonuclease or nickase. The Cas protein may be derived from the genus of microorganisms containing an ortholog of the Cas protein selected from the group consisting of, for example, Corynebacter, Sutterella, Legionella, Treponema, Filifactor, Eubacterium, Streptococcus (Streptococcus pyogenes), Lactobacillus, Mycoplasma, Bacteroides, Flaviivola, Flavobacterium, Azospirillum, Gluconacetobacter, Neisseria, Roseburia, Parvibaculum, Staphylococcus (Staphylococcus aureus), Nitratifractor, Corynebacterium, and Campylobacter, and may be simply isolated therefrom or recombinant.

The target-specific nuclease may be isolated from a microorganism or artificially or non-naturally occurring, obtained, for example, by a recombinant or synthetic method. In an embodiment, the target-specific nuclease (e.g. Cas9, Cpf1, etc.) may be a recombinant protein made by recombinant DNA. Recombinant DNA (rDNA) is a DNA molecule artificially created by a genetic recombination method such as molecular cloning to include heterologous or homologous genetic material obtained from various organisms. For example, when recombinant DNA is expressed in an appropriate organism to produce a target-specific nuclease (in vivo or in vitro), the recombinant DNA may have a nucleic acid sequence reconstructed by selecting a codon optimized for expression in the organism among codons encoding a protein to be produced.

The Cas protein may be included in a mutated form, which may mean that it is mutated to eliminate endonuclease activity that cleaves double-stranded DNA, and there is exemplified at least one selected from among mutation target-specific nucleases mutated to eliminate endonuclease activity and have nickase activity and forms mutated to eliminate both endonuclease activity and nickase activity.

When having nickase activity, a nick may be introduced to the strand where base conversion occurs or the opposite strand (e.g. the strand opposite the strand where base conversion occurs) (e.g. a nick is introduced at a position between the 3 rd nucleotide and the 4th nucleotide in a direction of the 5′ end of the PAM sequence on the strand opposite the strand where PAM is located). Such mutations (e.g. amino acid substitutions, etc.) may occur in a catalytically active domain (e.g. a RuvC catalytic domain in Cas9). Also, Streptococcus pyogenes-derived Cas9 may include mutations in which at least one selected from the group consisting of a catalytically active aspartate residue (aspartic acid at position 10 (D10), etc.), glutamic acid at position 762 (E762), histidine at position 840 (H840), asparagine at position 854 (N854), asparagine at position 863 (N863), aspartic acid at position 986 (D986), and the like is substituted with any different amino acid. Here, the different amino acid that is substituted may be alanine, but is not limited thereto.

In some cases, a Streptococcus pyogenes-derived Cas9 protein may be mutated to recognize NGA (in which N is any base selected from among A, T, G, and C) that is different from the PAM sequence (NGG) of wild-type Cas9 by substituting one or more selected from among aspartic acid at position 1135 (D1135), arginine at position 1335 (R1335), and threonine at position 1337 (T1337), for example, all three, with different amino acids.

For example, in the amino acid sequences of the Streptococcus pyogenes-derived Cas9 protein, amino acid substitution may occur at:
(1) D10, H840, or D10+H840;
(2) D1135, R1335, T1337, or D1135+R1335+T1337; or
(3) both residues (1) and (2).

Here, the “different amino acid” may be alanine, isoleucine, leucine, methionine, phenylalanine, proline, tryptophan, valine, asparagine, cysteine, glutamine, glycine, serine, threonine, tyrosine, aspartic acid, glutamic acid, arginine, histidine, or lysine, and may refer to an amino acid selected from among amino acids excluding amino acids at original mutation positions in a wild-type protein from all known variants of the amino acids described above. In an exemplary embodiment, the “different amino acid” may be alanine, valine, glutamine, or arginine. The different amino acid that is substituted may be, but is not limited to, alanine.

In an embodiment, the prime editor protein may be (1) a mixture of (i) a target-specific nuclease or a variant thereof and (ii) an HIV reverse transcriptase or a variant thereof; or (2) a fusion protein configured such that (ii) an HIV reverse transcriptase or a variant thereof is linked to the terminus of (i) a target-specific nuclease or a variant thereof. The nuclease or the variant thereof and the reverse transcriptase may be provided in the form in which a nuclease or a variant thereof and a reverse transcriptase are included individually, or in the form of a fusion protein of the nuclease or the variant thereof and the reverse transcriptase.

In the prime editor protein, the fusion protein may be configured such that (ii) the HIV reverse transcriptase or the variant thereof may bind to the N-terminus or C-terminus of the target-specific nuclease or the variant thereof. In a specific embodiment according to the present invention, the HIV reverse transcriptase or the variant thereof binds to the C-terminus of the target-specific nuclease or the variant thereof.

In some cases, when an HIV reverse transcriptase or a variant thereof is linked to a target-specific nuclease or a variant thereof, a linker may be used therefor. The linker may be exemplified by a peptide linker.

The peptide linker may be about 2-25aa long. Examples thereof may include, but are not limited to, amino acids such as alanine, glycine, and/or serine.

Examples of the linker may include (AnS)m (in which n and m are each 1 to 10), (GS)n, (GGS)n, (GSGGS)n, and (GnS)m (in which n and m are each 1 to 10), and the linker may be exemplified by (AnS)m or (GnS)m (in which n and m are each 1 to 10). Specifically, the linker may be (AnS)m in which n=1 and m=1, or G4S or (G4S)2 in which n=4 and m is 1 or 2 in (GnS)m.

In an embodiment, the HIV reverse transcriptase or the variant thereof may be at least one selected from the group consisting of:

HIV RT p51 of SEQ ID NO: 1;
PISPIETVPVKLKPGMDGPKVKQWPLTEEKIKALVEICTE
MEKEGKISKIGPENPYNTPVFAIKKKDSTKWRKLVDFREL
NKRTQDFWEVQLGIPHPAGLKQKKSVTVLDVGDAYFSVPL
DKDERKYTAFTIPSINNETPGIRYQYNVLPQGWKGSPAIF
QCSMTKILEPFRKQNPDIVIYQYMDDLYVGSDLEIGQHRT
KIEELRQHLLRWGFTTPDKKHQKEPPFLWMGYELHPDKWT
VQPIVLPEKDSWTVNDIQKLVGKLNWASQIYAGIKVRQLC
KLLRGTKALTEVVPLTEEAELELAENREILKEPVHGVYYD
PSKDLIAEIQKQGQGQWTYQIYQEPFKNLKTGKYARMKGA
HTNDVKQLTEAVQKIATESIVIWGKTPKFKLPIQKETWEA
WWTEYWQATWIPEWEFVNTPPLVKLWYQLEKEPIIGAETF
HIV RT p66 of SEQ ID NO: 2;
PISPIETVPVKLKPGMDGPKVKQWPLTEEKIKALVEICTE
MEKEGKISKIGPENPYNTPVFAIKKKDSTKWRKLVDFREL
NKRTQDFWEVQLGIPHPAGLKQKKSVTVLDVGDAYFSVPL
DKDFRKYTAFTIPSINNETPGIRYQYNVLPQGWKGSPAIF
QCSMTKILEPFRKQNPDIVIYQYMDDLYVGSDLEIGQHRT
KIEELRQHLLRWGFTTPDKKHQKEPPFLWMGYELHPDKWT
VQPIVLPEKDSWTVNDIQKLVGKLNWASQIYAGIKVRQLC
KLLRGTKALTEVVPLTEEAELELAENREILKEPVHGVYYD
PSKDLIAEIQKQGQGQWTYQIYQEPFKNLKTGKYARMKGA
HTNDVKQLTEAVQKIATESIVIWGKTPKFKLPIQKETWEA
WWTEYWQATWIPEWEFVNTPPLVKLWYQLEKEPIIGAETF
YVDGAANRETKLGKAGYVTDRGRQKVVPLTDTTNQKTELQ
AIHLALQDSGLEVNIVTDSQYALGIIQAQPDKSESELVSQ
IIEQLIKKEKVYLAWVPAHKGIGGNEQVDGLVSAGIRKVL
an HIV RT p51 variant of
SEQ ID NO: 3 (p51(2));
and
PISPIETVPVKLKPGMDGPKVKQWPLTEEKIKALVEICTE
MEKEGKISKIGPENPYNTPVFAIKKKDSTKWRKLVDFREL
NKRTQDFWEVQLGIPHPAGLKKKKSVTVLDVGDAYFSVPL
DEDERKYTAFTIPSINNETPGIRYQYNVLPQGWKGSPAIF
QSSMTKILEPFRKQNPDIVIYQYMDDLYVGSDLEIGQHRT
KIEELRQHLLRWGLTTPDKKHQKEPPFLWMGYELHPDKWT
VQPIVLPEKDSWTVNDIQKLVGKLNWASQIYPGIKVRQLC
KLLRGTKALTEVIPLTEEAELELAENREILKEPVHGVYYD
PSKDLIAEIQKQGQGQWTYQIYQEPFKNLKTGKYARMRGA
HINDVKQLTEAVQKITTESIVIWGKTPKFKLPIQKETWET
WWTEYWQATWIPEWEFVNTPPLVKLWYQLEKEPIVGAETF
an HIV RT p66 variant of
SEQ ID NO: 4 (p66(2)).
PISPIETVPVKLKPGMDGPKVKQWPLTEEKIKALVEICTE
MEKEGKISKIGPENPYNTPVFAIKKKDSTKWRKLVDFREL
NKRTQDFWEVQLGIPHPAGLKKKKSVTVLDVGDAYFSVPL
DEDERKYTAFTIPSINNETPGIRYQYNVLPQGWKGSPAIF
QSSMTKILEPFRKQNPDIVIYQYMDDLYVGSDLEIGQHRT
KIEELRQHLLRWGLITPDKKHQKEPPFLWMGYELHPDKWT
VQPIVLPEKDSWTVNDIQKLVGKLNWASQIYPGIKVRQLC
KLLRGTKALTEVIPLTEEAELELAENREILKEPVHGVYYD
PSKDLIAEIQKQGQGQWTYQIYQEPFKNLKTGKYARMRGA
HINDVKQLTEAVQKITTESIVIWGKIPKFKLPIQKETWET
WWTEYWQATWIPEWEFVNTPPLVKLWYQLEKEPIVGAETF
YVDGAANRETKLGKAGYVINRGRQKVVILIDITNQKTELQ
AIYLALQDSGLEVNIVTDSQYALGIIQAQPDQSESELVNQ
IIEQLIKKEKVYLAWVPAHKGIGGNEQVDKLVSAGIRKVL

In some cases, when any one of the foregoing is selected as the HIV reverse transcriptase or the variant thereof, an HIV reverse transcriptase or a variant thereof different from ii) the HIV reverse transcriptase or the variant thereof that is selected may be further included.

The HIV reverse transcriptase or the variant thereof may be linked to the target-specific nuclease or the variant thereof to form a fusion protein, or may be included separately without being linked to the target-specific nuclease or the variant thereof.

Specifically, when HIV RT p51 of SEQ ID NO: 1 is selected, at least one selected from the group consisting of HIV RT p66 of SEQ ID NO: 2, an HIV RT p51 variant of SEQ ID NO: 3, an HIV RT p66 variant of SEQ ID NO: 4 may be further included.

When HIV RT p66 of SEQ ID NO: 2 is selected, at least one selected from the group consisting of HIV RT p51 of SEQ ID NO: 1, an HIV RT p51 variant of SEQ ID NO: 3, and an HIV RT p66 variant of SEQ ID NO: 4 may be further included.

When the HIV RT p51 variant of SEQ ID NO: 3 is selected, at least one selected from the group consisting of HIV RT p51 of SEQ ID NO: 1, HIV RT p66 of SEQ ID NO: 2, and an HIV RT p66 variant of SEQ ID NO: 4 may be further included.

When the HIV RT p66 variant of SEQ ID NO: 4 is selected, at least one selected from the group consisting of HIV RT p51 of SEQ ID NO: 1, HIV RT p66 of SEQ ID NO: 2, and an HIV RT p51 variant of SEQ ID NO: 3 may be further included.

In a specific embodiment according to the present invention, genome editing was confirmed in the human cell line genome by a fusion protein in which the HIV RT p51 or p66 subunit was linked to the Cas9 H840A nickase C-term.

When HIV p66 was additionally expressed in addition to the H840A-HIV p51 fusion protein or when HIV p51 was additionally expressed in addition to the H840A-HIV p66 fusion protein, genome editing efficiency was confirmed to greatly increase compared to when either one of the two HIV subunits linked to H840A was expressed alone.

Also, it was confirmed that the genome editing efficiency of HIV RT p66(2) was higher than that of HIV RT p66.

The nucleic acid encoding the prime editor protein may include a nucleic acid encoding (i) the target-specific nuclease or the variant thereof and a nucleic acid encoding (ii) the HIV reverse transcriptase or the variant thereof.

The prime editing guide RNA includes an editing sequence and functions as a reverse transcriptase template. The reverse transcriptase (RT) is an RNA-dependent DNA polymerase capable of synthesizing a DNA strand (i.e. complementary DNA, cDNA) using a reverse transcriptase template.

The prime editing guide RNA (pegRNA) or DNA encoding the same includes a binding site that binds to a genome to be edited and an editing sequence.

The sequence including the editing sequence serves as a reverse transcriptase template. The reverse transcriptase template includes a desired editing sequence and is homologous to the genomic DNA locus. The editing sequence is a heterologous sequence and includes a target sequence to be edited in the genome.

The binding site may be located arbitrarily in the 5′ direction or the 3′ direction of the reverse transcriptase template, and particularly, the binding site may be located in the 3′ direction of the reverse transcriptase template.

The binding site may include a sequence complementary to a genomic DNA strand nicked by a nuclease included in the prime editor protein or a variant thereof, for example, by a nickase. The binding site may be a target site that hybridizes to a target site and serves as an initiation point for activating the reverse transcriptase.

The binding site may include 5 or more, 6 or more, 7 or more, 8 or more, 9 or more, 10 or more, 11 or more, 12 or more, 13 or more, 14 or more, 15 or more, 20 or more, or 25 or more nucleotides with at least 80%, for example, at least 85%, at least 90%, at least 95%, at least 97%, at least 98%, at least 99%, or 100% homology to the sequence of the target site.

The composition according to the present invention includes (1) the prime editor protein including (i) the target-specific nuclease or the variant thereof and (ii) the HIV (human immunodeficiency virus) reverse transcriptase (RT) or the variant thereof, or the nucleic acid encoding the same, and (2) the prime editing guide RNA (pegRNA) including the binding site that binds to a genome to be edited and the editing sequence, and in order to deliver (1) and (2), a single delivery vehicle or a plurality of delivery vehicles may be used in combination with the same or different configurations.

The (1) may be included in the first delivery vehicle, and the (2) may be included in the second delivery vehicle, and these two delivery systems may be viral delivery vehicles simultaneously, may be a viral delivery vehicle and a non-viral delivery vehicle respectively, or may be non-viral delivery vehicles simultaneously.

The nucleic acid may be an RNA sequence, a DNA sequence, or a combination thereof (RNA-DNA hybrid sequence). The prime editing guide RNA may include an RNA sequence of guide RNA or a DNA sequence encoding the same.

A DNA sequence encoding the prime editor protein of (1) and a DNA sequence encoding the prime editing guide RNA of (2) may be provided through a delivery vehicle such as a vector. The DNA sequence encoding the (1) and the DNA sequence encoding the (2) may be placed on the same vector and delivered simultaneously through one vector. The DNA sequence encoding the prime editor protein of (1) and the DNA sequence encoding the prime editing guide RNA of (2) may be separately placed on different vectors and delivered.

The composition according to the present invention may be delivered using a viral vector such as adeno-associated viral vector (AAV), adenoviral vector (AdV), lentiviral vector (LV), or retroviral vector (RV), and other viral vectors such as episomal vectors including Simian virus 40 (SV40) ori, bovine papilloma virus (BPV) ori, or Epstein-Barr nuclear antigen (EBV) ori.

The vector may be delivered in vivo or into cells through microinjection (e.g. direct injection into a lesion or target site), electroporation, lipofection, viral vector, nanoparticles, PTD (protein translocation domain) fusion protein method, etc.

In some cases, a DNA sequence encoding the prime editing guide of (2) may be delivered through a vector. The prime editor protein of (1) or an RNA sequence encoding the same may be delivered in the form of mRNA. The prime editor protein or mRNA may be delivered directly or through a carrier.

Also, an RNA sequence encoding the prime editor protein of (1) and an RNA sequence encoding the prime editing guide of (2) may be included. Here, mRNA encoding the (1) and mRNA of (2) may be delivered. The mRNA may be delivered directly or through a carrier.

Furthermore, the prime editor protein of (1) and mRNA of the prime editing guide RNA of (2) may form an assembled ribonucleoprotein (RNP) complex and deliver the same. The RNP may be delivered directly or through a carrier.

Since RNP is normally degraded in vivo within 72 hours, it is unlikely to remain continuously and cause toxicity and off-target editing, which is advantageous when used in gene therapy. PE may also be introduced into eukaryotic cells in the form of plasmid DNA rather than RNP, but in this case, plasmid fragments may be inserted into the genome. In particular, for gene editing in plant cells, the RNP method may not be subject to GMO regulations unlike the DNA method.

The RNP complex may be delivered to cells through various methods in the art, such as microinjection, electroporation, DEAE-dextran treatment, lipofection, nanoparticle-mediated transfection, protein transduction domain-mediated introduction, and PEG-mediated transfection, but the present invention is not limited thereto.

Examples of the carrier may include, but are not limited to, a cell penetrating peptide (CPP), nanoparticles, and a polymer. CPP is a short peptide that facilitates cellular uptake of various molecular cargoes (from nanoscale particles to small chemical molecules and large fragments of DNA). The cargo may include (1) the prime editor protein or the nucleic acid encoding the same and (2) the prime editing guide RNA. (1) The prime editor protein or the nucleic acid encoding the same may be assembled through chemical bonding through covalent bonding or non-covalent interaction. (2) The prime editing guide RNA or a polynucleotide encoding the same is complexed with CPP to form condensed positively charged particles.

Regarding the nanoparticles, the composition according to the present invention may be delivered via polymer nanoparticles, metal nanoparticles, metal/inorganic nanoparticles, or lipid nanoparticles. The polymer nanoparticles may be, for example, DNA nanoclews or thread-like DNA nanoparticles synthesized by rolling circle amplification. DNA nanoclews or thread-like DNA nanoparticles may be loaded with (1) the prime editor protein or the nucleic acid encoding the same and (2) the prime editing guide RNA, and coated with PEI to improve endosomal escape capacity. These complexes bind to cell membranes, are internalized, and then move to the nucleus through endosomal escape, allowing simultaneous delivery of (1) and (2).

Regarding the metal nanoparticles, gold particles may be linked to (1) the prime editor protein or the nucleic acid encoding the same and (2) the prime editing guide RNA, and complexed with a cationic endosomal disruptive polymer and thus delivered to cells. Examples of the cationic endosomal disruptive polymer may include polyethylene imine, poly(arginine), poly(lysine), poly(histidine), poly-[2-{(2-aminoethyl)amino}-ethyl-aspartamide (pAsp(DET)), block copolymer of poly(ethylene glycol) (PEG) and poly(arginine), block copolymer of PEG and poly(lysine), and block copolymer of PEG and poly{N-[N-(2-aminoethyl)-2-aminoethyl]aspartamide} (PEG-pAsp(DET)).

Regarding the metal/inorganic nanoparticles, ZIF-8 (zeolitic imidazolate framework-8) may be used to encapsulate (1) the prime editor protein or the nucleic acid encoding the same and (2) the prime editing guide RNA, and negatively charged RNP may be encapsulated with positively charged nanoscale ZIF. Efficient endosomal escape is capable of altering the expression of target genes of interest.

DNA or nucleic acids encoding the negatively charged (1) and (2) may be coupled with cationic materials to form nanoparticles, which may penetrate cells through receptor-mediated endocytosis or phagocytosis. The RNP complex of (1) and (2) may bind to a cationic polymer. Examples of the cationic polymer may include polyallylamine (PAH); polyethyleneimine (PEI); poly(L-lysine) (PLL); poly(L-arginine) (PLA); polyvinylamine homopolymers or copolymers; poly(vinylbenzyl-tri-C1-C4-alkylammonium salts); polymers of aliphatic or alicyclic dihalides and aliphatic N,N,N′,N′-tetra-C1-C4-alkyl-alkylenediamines; poly(vinylpyridine) or poly(vinylpyridinium salt); poly(N,N-diallyl-N,N-di-C1-C4-alkyl-ammonium halide); homopolymers or copolymers of quaternized di-C1-C4-alkyl-aminoethyl acrylates or methacrylates; POLYQUAD™; polyaminoamide, and the like.

The cationic lipids may include cationic liposomal formulations. The lipid bilayer of liposomes may protect encapsulated nucleic acids from degradation and may prevent specific neutralization by antibodies capable of binding to nucleic acids. During endosomal maturation, endosome membranes and liposomes are fused, enabling efficient endosomal escape of cationic lipid-nucleases. Examples of the cationic lipids may include polyethyleneimine, starburst polyamidoamine (PAMAM) dendrimers, Lipofectin (combination of DOTMA and DOPE), lipofectase, LIPOFECTAMINE® (e.g. Lipofectamine® 2000, Lipofectamine® 3000, Lipofectamine® RNAiMAX, Lipofectamine® LTX), SAINT-RED (Synvolux Therapeutics, Groningen, The Netherlands), DOPE, Cytofectin (Gilead Sciences, Foster City, California), and Eufectin (JBL, San Luis Obispo, California). Representative cationic liposomes may be prepared from N-[1-(2,3-dioleyloxy)-propyl]-N,N,N-trimethylammonium chloride (DOTMA), N-[1-(2,3-dioleoyloxy)-propyl]-N,N,N-trimethylammonium methyl sulfate (DOTAP), 3β-[N-(N′,N′-dimethylaminoethane)carbamoyl]cholesterol (DC-Chol), 2,3-dioleyloxy-N-[2-(sperminecarboxamido)ethyl]-N,N-dimethyl-1-propanaminium trifluoroacetate (DOSPA), 1,2-dimyristyloxypropyl-3-dimethyl-hydroxyethyl ammonium bromide, or dimethyldioctadecylammonium bromide (DDRB).

Regarding the lipid nanoparticles, delivery is possible using liposomes as carriers. Liposomes are spherical vesicular structures composed of a unilamellar or multilamellar lipid bilayer surrounding an inner aqueous compartment and a relatively impermeable outer lipophilic phospholipid bilayer. Liposomal formulations may primarily contain natural phospholipids and lipids such as 1,2-distearoyl-sn-glycero-3-phosphatidylcholine (DSPC), sphingomyelin, phosphatidylcholine, or monosialoganglioside. In some cases, cholesterol or 1,2-dioleoyl-sn-glycero-3-phosphoethanolamine (DOPE) may be added to the lipid membrane in order to resolve instability in plasma. The addition of cholesterol reduces the rapid release of encapsulated bioactive compounds into plasma or 1,2-dioleoyl-sn-glycero-3-phosphoethanolamine (DOPE) increases stability.

Another aspect of the present invention pertains to a prime editing method including treating cells with the composition described above.

The cells may include, but are not limited to, eukaryotic cells (e.g. fungi such as yeast, eukaryotic animal and/or eukaryotic plant-derived cells (e.g. embryonic cells, stem cells, somatic cells, germ cells, etc.), etc.), eukaryotic animals (e.g. primates such as humans, monkeys, dogs, pigs, cattle, sheep, goats, mice, rats, etc.), or eukaryotic plants (e.g. algae such as green algae, corn, soybeans, wheat, rice, etc.).

### Examples

A better understanding of the present invention may be obtained through the following examples. These examples are merely set forth to illustrate the present invention, and are not to be construed as limiting the scope of the present invention, as will be apparent to those skilled in the art.

### Example 1

In order to convert a GFP phenotype to a BFP phenotype by editing the CTT sequence (base sequence encoding the 65th and 66th amino acid regions) of a GFP gene to GCC, various versions of HIV reverse transcriptase-based prime editors were expressed in GFP stable cells, after which the corresponding fluorescence signal was analyzed using FACS equipment.

The prime editors thus constructed were as follows:
Mock: original HEK293-gfp stable cell without gene expression
PE only: Expression of existing H840A-MMLV RT fusion protein alone (without pegRNA)
HIVPE-66 only: Expression of H840A-HIV RT p66 fusion protein alone (without pegRNA)
HIVPE-66(2) only: Expression of H840A-HIV RT p66(2) fusion protein alone (without pegRNA)
PE+peg: Expression of existing H840A-MMLV RT fusion protein and pegRNA
HIVPE-51+peg: Expression of H840A-HIV RT p51 fusion protein and pegRNA
HIVPE-66+peg: Expression of H840A-HIV RT p66 fusion protein and pegRNA
HIVPE-51(2)+peg: Expression of H840A-HIV RT p51(2) fusion protein and pegRNA
HIVPE-66(2)+peg: Expression of H840A-HIV RT p66(2) fusion protein and pegRNA
PE+Cont.Vec+peg: Expression of existing H840A-MMLV RT fusion protein, control vector, and pegRNA
HIVPE-51+66+peg: Expression of H840A-HIV RT p51 fusion protein, HIV p66 (not fusion protein), and pegRNA
HIVPE-66+51+peg: Expression of H840A-HIV RT p66 fusion protein, HIV p51 (not fusion protein), and pegRNA
HIVPE-51(2)+66(2)+peg: Expression of H840A-HIV RT p51(2) fusion protein, HIV p66(2) (not fusion protein), and pegRNA
HIVPE-66(2)+51(2)+peg: Expression of H840A-HIV RT p66(2) fusion protein, HIV p51(2) (not fusion protein), and pegRNA.

HIV RT p51 of SEQ ID NO: 1;
PISPIETVPVKLKPGMDGPKVKQWPLTEEKIKALVEICTE
MEKEGKISKIGPENPYNTPVFAIKKKDSTKWRKLVDFREL
NKRTQDFWEVQLGIPHPAGLKQKKSVTVLDVGDAYFSVPL
DKDFRKYTAFTIPSINNETPGIRYQYNVLPQGWKGSPAIF
QCSMTKILEPFRKQNPDIVIYQYMDDLYVGSDLEIGQHRT
KIEELRQHLLRWGFTTPDKKHQKEPPFLWMGYELHPDKWT
VQPIVLPEKDSWTVNDIQKLVGKLNWASQIYAGIKVRQLC
KLLRGTKALTEVVPLTEEAELELAENREILKEPVHGVYYD
PSKDLIAEIQKQGQGQWTYQIYQEPFKNLKTGKYARMKGA
HTNDVKQLTEAVQKIATESIVIWGKTPKFKLPIQKETWEA
WWTEYWQATWIPEWEFVNTPPLVKLWYQLEKEPIIGAETF
HIV RT p66 of SEQ ID NO: 2;
PISPIETVPVKLKPGMDGPKVKQWPLTEEKIKALVEICTE
MEKEGKISKIGPENPYNTPVFAIKKKDSTKWRKLVDFREL
NKRTQDFWEVQLGIPHPAGLKQKKSVTVLDVGDAYFSVPL
DKDERKYTAFTIPSINNETPGIRYQYNVLPQGWKGSPAIF
QCSMTKILEPFRKQNPDIVIYQYMDDLYVGSDLEIGQHRT
KIEELRQHLLRWGFTTPDKKHQKEPPFLWMGYELHPDKWT
VQPIVLPEKDSWTVNDIQKLVGKLNWASQIYAGIKVRQLC
KLLRGTKALTEVVPLTEEAELELAENREILKEPVHGVYYD
PSKDLIAEIQKQGQGQWTYQIYQEPFKNLKTGKYARMKGA
HTNDVKQLTEAVQKIATESIVIWGKTPKFKLPIQKETWEA
WWTEYWQATWIPEWEFVNTPPLVKLWYQLEKEPIIGAETF
YVDGAANRETKLGKAGYVTDRGRQKVVPLTDTTNQKTELQ
AIHLALQDSGLEVNIVTDSQYALGIIQAQPDKSESELVSQ
IIEQLIKKEKVYLAWVPAHKGIGGNEQVDGLVSAGIRKVL
HIV RT p51 variant of SEQ ID NO: 3 (p51(2));
and
PISPIETVPVKLKPGMDGPKVKQWPLTEEKIKALVEICTE
MEKEGKISKIGPENPYNTPVFAIKKKDSTKWRKLVDFREL
NKRTQDFWEVQLGIPHPAGLKKKKSVTVLDVGDAYFSVPL
DEDERKYTAFTIPSINNETPGIRYQYNVLPQGWKGSPAIF
QSSMIKILEPFRKQNPDIVIYQYMDDLYVGSDLEIGQHRT
KIEELRQHLLRWGLITPDKKHQKEPPFLWMGYELHPDKWT
VQPIVLPEKDSWTVNDIQKLVGKLNWASQIYPGIKVRQLC
KLLRGTKALTEVIPLTEEAELELAENREILKEPVHGVYYD
PSKDLIAEIQKQGQGQWTYQIYQEPFKNLKTGKYARMRGA
HTNDVKQLTEAVQKITTESIVIWGKTPKFKLPIQKETWET
WWTEYWQATWIPEWEFVNTPPLVKLWYQLEKEPIVGAETF
HIV RT p66 variant of SEQ ID NO: 4 (p66(2)).
PISPIETVPVKLKPGMDGPKVKQWPLTEEKIKALVEICTE
MEKEGKISKIGPENPYNTPVFAIKKKDSTKWRKLVDFREL
NKRTQDFWEVQLGIPHPAGLKKKKSVTVLDVGDAYFSVPL
DEDFRKYTAFTIPSINNETPGIRYQYNVLPQGWKGSPAIF
QSSMTKILEPFRKQNPDIVIYQYMDDLYVGSDLEIGQHRT
KIEELRQHLLRWGLTTPDKKHQKEPPFLWMGYELHPDKWT
VQPIVLPEKDSWTVNDIQKLVGKLNWASQIYPGIKVRQLC
KLLRGTKALTEVIPLTEEAELELAENREILKEPVHGVYYD
PSKDLIAEIQKQGQGQWTYQIYQEPFKNLKIGKYARMRGA
HINDVKQLTEAVQKITTESIVIWGKTPKFKLPIQKETWET
WWTEYWQATWIPEWEFVNTPPLVKLWYQLEKEPIVGAETF
YVDGAANRETKLGKAGYVINRGRQKVVTLTDTTNQKTELQ
AIYLALQDSGLEVNIVTDSQYALGIIQAQPDQSESELVNQ
IIEQLIKKEKVYLAWVPAHKGIGGNEQVDKLVSAGIRKVL

The results thereof are shown in FIG. 1. As shown in FIG. 1, (a) in the negative control experimental groups without pegRNA, the percentage of BFP positive cells was determined to be 0% (red part). (b) When MMLV-based PE (PE+peg experimental group) was expressed in the presence of pegRNA, the percentage of BFP positive cells was determined to be 39.9%. When PE using HIV RT p51 or p66 was expressed along with pegRNA, the percentage of BFP positive cells was determined to be 3.2%-15.1%, and the percentage of BFP positive cells was determined to be higher when using the p66 subunit rather than p51. (c) When p51 and p66 were simultaneously expressed, the percentage of BFP positive cells was determined to be higher (22.4-29.5%) than when individually expressed (b).

The CTT-to-GCC editing efficiency induced by HIV RT-based PE in the human cell line (HEK-293 GFP stable cell) was determined through next generation sequencing (NGS) at the genome level. The results thereof are shown in FIG. 2.

As shown in FIG. 2, when PE using HIV RT p51 was expressed along with pegRNA, the GCC editing efficiency was determined to be 3.96-9.92%, and when PE using the p66 subunit was expressed along with pegRNA, the GCC editing efficiency was determined to be 16.68-21.30%. When both HIV subunits were co-expressed, the GCC editing efficiency was determined to be 29.49-34.01%. These results showed a pattern matching the level of BFP positive cells (phenotype results) described above.

Although specific embodiments of the present invention have been disclosed in detail above, it will be obvious to those skilled in the art that the description is merely of preferable exemplary embodiments and is not to be construed as limiting the scope of the present invention. Therefore, the substantial scope of the present invention will be defined by the appended claims and equivalents thereto.

### [Sequence List Free Text]

An electronic file is attached.

1. A composition for prime editing comprising a prime editor protein or a nucleic acid encoding the prime editor protein and a prime editing guide RNA (pegRNA), wherein the prime editor protein comprises (i) a target-specific nuclease or a variant thereof and (ii) an HIV (human immunodeficiency virus) reverse transcriptase (RT) or a variant thereof.

2. The composition according to claim 1, wherein the prime editor protein is (1) configured such that (i) the target-specific nuclease or the variant thereof and (ii) the HIV reverse transcriptase or the variant thereof are individually contained; or is (2) a fusion protein in which (ii) the HIV reverse transcriptase or the variant thereof is linked to a terminus of the target-specific nuclease or the variant thereof.

3. The composition according to claim 1, wherein the nucleic acid encoding the prime editor protein comprises a nucleic acid encoding (i) the target-specific nuclease or the variant thereof and a nucleic acid encoding (ii) the HIV reverse transcriptase or the variant thereof.

4. The composition according to claim 3, wherein the nucleic acid encoding (i) the target-specific nuclease or the variant thereof or the nucleic acid encoding (ii) the HIV RT or the variant thereof and the prime editing guide RNA are inserted into identical or different vectors.

5. The composition according to claim 3, comprising a vector comprising, separately or together, the nucleic acid encoding the prime editor protein and a nucleic acid encoding the prime editing guide RNA.

6. The composition according to claim 3, comprising a vector comprising nucleic acids encoding the prime editor protein and the prime editing guide RNA.

7. The composition according to claim 1, wherein the target-specific nuclease is Cas9.

8. The composition according to claim 1, wherein the variant of the target-specific nuclease is configured such that at least one amino acid selected from the group consisting of D10, E762, H839, H840, N854, N863, and D986 of Cas9 is substituted with a different amino acid.

9. The composition according to claim 8, wherein the different amino acid is alanine.

10. The composition according to claim 1, wherein the HIV reverse transcriptase or the variant thereof is at least one selected from the group consisting of:
HIV RT p51 of SEQ ID NO: 1;
HIV RT p66 of SEQ ID NO: 2;
an HIV RT p51 variant of SEQ ID NO: 3; and
an HIV RT p66 variant of SEQ ID NO: 4.

11. The composition according to claim 1, further comprising an HIV reverse transcriptase or a variant thereof different from (ii) the HIV reverse transcriptase or the variant thereof.

12. A prime editing method comprising treating cells with the composition according to claim 1.
